(19) United States
(12) Patent Application Publication
(10) Pub. No.: US 2012/0071717 A1
(43) Pub. Date: Mar. 22, 2012

(54) Dressing and method for cleaning and protecting a laparoscopic camera lens intra-abdominally, stomas, appendages, and insertion sites of tubes and catheters

(75) Inventors: Suzanne Podolski, Princeton, NJ (US); Andrew Camerota, Princeton, NJ (US)

(73) Assignee: Abdominal Devices, LLC, Princeton, NJ (US)

(21) Appl. No.: 13/200,052

(22) Filed: Sep. 16, 2011

Related U.S. Application Data
(60) Provisional application No. 61/403,558, filed on Sep. 17, 2010.

(51) Int. Cl. A61F 13/45; A61B 1/04

(52) U.S. Cl. 600/109; 604/369

(57) ABSTRACT

A dressing and method for cleaning a laparoscopic camera lens during surgical procedures when placed around prior art or grasped by prior art and then introduced into the abdominal cavity of a patient and then manipulated against a laparoscopic camera lens cleaning and defogging the lens providing an improved field of site. Further, a dressing and method for protecting and cleaning stomas, appendages, and the insertion sites of tubes and catheters in patients. An annular shaped medical grade polyurethane hydrophilic or similar foam like dressing is placed around a stoma, or at the insertion site of a catheter or tube creating a protective seal inhibiting bacterium from entering the body, further providing cushioning and absorption of human fluid that can cause skin breakdown and infection, increasing the life of stoma appliances, tubes and catheters, providing patients additional comfort, and when placed around an appendage provides effective wound healing.

### BACKGROUND OF THE INVENTION

### Field of the Invention

The present invention relates to a medical-surgical dressing and its method of use. More particularly, this invention relates to a medical grade polyurethane hydrophilic foam or sponge like ring dressing specially suited for use with laparoscopic surgical equipment during a laparoscopic and or a robotic surgical procedure, ostomy appliances, around a stoma or appendage, and at the insertion cites of tubes and catheters in patients. The present invention is designed to absorb various bodily fluids, odors, and gases, reduce patient discomfort, protect the skin, and provide a protective barrier from bacteria as well as other foreign bodies and fluids at the insertion site of tubes and catheters. Further, the present invention is designed to extend the useful service life of ostomy appliances as well as tubes and catheters. Additionally, the present invention is designed to provide an effective method for cleaning a stoma or the insertion site of a tube or catheter effectively with little irritation to the patient while protecting the skin. Further, the present invention provides an effective method for cleaning laparoscopic equipment during laparoscopic surgery. The dressing can be made or used with anti-bacterial agents, x-ray detectable elements, as well as fragrance.

There are many intestinal and urinary diseases which are treated by surgical rerouting of waste from a patients usual waste elimination route. Among the more common types of such procedures are colostomy, ileostomy and urostomy. These surgical procedures allow waste material to exit via an artificial opening, a stoma. Patients lose voluntary control over his or her elimination function. Therefore, an appliance is worn at all times to collect the waste materials exiting the body through the stoma.

A typical ostomy appliance system consists of a collection bag attached to a device that is of an adhesive material that is adhered to the skin of a patient. However, chronic liquid stool, or human fluid, seeps through the area where the bag and adhesive material connect. Further, acid of human waste material breaks down the adhesive material and the entire ostomy system can fall off of the patient. Further, gas omitted from the stoma site often inflates the collection bag to the extent it has to be removed or relieved, omitting a foul odor that is embarrassing to the patient and unpleasant.

Other ostomy appliances are designed to cover the stoma completely allowing the patient to remove the appliance and human waste after a period of time. However, there is leakage around the connection site of the device to the patient. Patients with ileostomies and urostomies cannot use this type of device due to chronic liquid waste. Further, gas from the stoma releases directly from the device omitting a foul odor that is embarrassing to the patient and unpleasant.

Typically, the useful service of the life of the above-mentioned ostomy appliances will be a few hours to four days depending on many factors. Some of the factors include activity of the patient, weather, perspiration of the patient, size, constancy and acidity of waste material, infection at the stoma site, and irritation of the skin. Use and frequent replacement of ostomy appliances are a continuous requirement in the life of the patient. Further, frequent cleaning of the stoma is a continuous requirement in the life of the patient. Current cleaning of stoma involves using cotton swabs to wipe in-between the stoma and skin and or using several gauze pads to wipe the area. These methods are messy, painful, and irritate the skin. Further, despite frequent cleaning, skin breakdown and infection are common occurrences for colostomy, urostomy, and ileostomy patients. It is the major goal of the present dressing and method to add to the useful life of each ostomy appliance, decrease emanating odor, irritation and infection at the stoma site, prevent skin breakdown, and provide a method of cleaning and protection that is more effective and less irritating than current methods.

The surgical performance of a tracheotomy has become increasingly necessary due to disease and related breathing difficulties. As a result of this procedure, a patient receives a stoma at the anterior lower neck, through which subsequent respiration takes place. There are several types of tubes and devices that are inserted into or around this type of stoma. Further, the character or variety of human fluid which is often expelled through the stoma by indicates infectious activity and skin irritation.

Current dressings, devices and materials used to collect and clean the human fluid around the stoma site of a tracheotomy are bulky in design or cumbersome to wear. Current methods for collecting human fluid involve taking gauze pads and wrapping them around the tubes inserted into the stoma, or any other device around the stoma. These pads get tangled, saturated with fluid, and become difficult to remove.

Use and frequent replacement of tracheotomy appliances are a continuous requirement in the life of the patient. Further, frequent cleaning of the stoma is a continuous requirement in the life of the patient. Infection, skin breakdown, and granulomas are common complications associated with this type of stoma. It is the major goal of the present dressing and method to add to the useful life of each tracheotomy appliance, decrease irritation and infection at the stoma site, prevent skin breakdown, and provide a method of cleaning and protection that is more effective and less irritating that current methods.

There are many medical-surgical uses of catheters and tubes in patients. Many patients due to illness, disease and or trauma have a tube inserted through their nasal passageway to collect gastric fluids and gas. Further, tubes inserted into the nasal passage are often replaced as the secretions of the nasal passage dissolve current tapes and anchoring devices used to secure the tube in place. The constant re-insertion of the nasal gastric tube subjects the patient to complications, X-Ray, and it is extremely uncomfortable. It is also costly for healthcare providers to spend more time and money for reinsertion. It is a major goal of the present dressing and method to add to the useful life of each nasogastric tube and decrease irritation at the insertion site.

Many patients due to illness, disease and or trauma have catheters inserted into their urethra to collect human waste. Infection and irritation at the insertion site is common. In addition, the human waste secreting around the insertion site causes breakdown of skin and genital tissue. Further, a bacterium that grows at the insertion site can move along the catheter into the bladder, then into the blood stream causing sepsis and death. For bedridden incontinent patients, stool seeps into the insertion site of a catheter causing infection, illness, and death. As a result of infection and irritation, urinary catheters are often replaced, which is costly to the healthcare provider as well as extremely uncomfortable for the patient. Frequent replacement of catheters is a requirement for patients. Further, frequent cleaning of the insertion site is a continuous requirement in the life of the patient. Current methods for cleaning the area involve washing the area with a wash cloth and cleaning material. However, for trauma patients or contracted elderly patients it is difficult to maneuver the body for proper cleaning. Further, if the patient is not properly cleaned and dried of cleaning material, it can cause further irritation to compromised skin. It is the major goal of the present dressing and method to add to the useful life of each catheter, as well as decrease irritation and infection at the insertion site of catheters, and the genital area, by providing a protective seal and effective method for cleaning.

Many patients due to illness, disease and or trauma, have a tube inserted into their rectum for the collection of stool. The skin at the insertion sites of rectal tubes becomes irritated due to leakage around the insertion site. As a result, rectal tissue is broken down causing bleeding, and infection. Further, for bedridden patients, the human waste leakage gets into the genital area of patients, which may cause urinary tract infections. Frequent replacement of rectal tubes is a continuous requirement. Further, frequent cleaning of the insertion site is a continuous requirement. Current methods to seal the insertion site include padding the buttocks with gauze or cloth. Gauze and cloth often dries to the skin and is difficult to remove causing further breakdown of a larger area of skin. It is the major goal of the present dressing and method to absorb human leakage around the insertion site of a rectal tube and or chronic anal leakage around the anus.

Many patients due to illness, disease and or trauma, have a tube inserted into their abdomen for the collection of fluid post-surgically, which attaches to a bulb for suctioning abdominal fluid. Further, many patients due to illness, disease and or trauma, have a feeding tube inserted into their abdomen for providing nourishment into the stomach. The skin at the insertion site of these types of tubes often become irritated and infected from human fluid leakage around and into the insertion site. This is particularly dangerous as colonized bacteria can travel along the tube into the stomach of the patient. Further, leakage causes skin breakdown around the site. These types of tubes are replaced often, which is timely, costly as well as extremely dangerous and uncomfortable for the patient. Use and frequent replacement of such tubes are a continuous requirement in the life of the patient. Further, frequent cleaning of the insertion site is a continuous requirement in the life of the patient. Current dressings and methods involve cutting square pads made of foam and or gauze. Gauze dries to the skin and becomes difficult to remove. Current foam dressings are not designed to fit around the tube at the insertion site so they are ineffective for protecting the insertion site, which is still vulnerable to colonization of bacterium. Further, current methods for cleaning this type of site involve using a cotton swab to wipe in-between the tube and the skin at the insertion site, which is painful, and damaging to the integrity of the skin. It is the major goal of the present dressing and method to add to the useful life of each tube and catheter, as well as decrease skin breakdown, irritation, and infection at the insertion site providing a protective seal and effective method of cleaning.

Many patients due to illness, disease and or trauma get a wound or callous around an appendage such as a toe or finger. Current dressings for fingers and toes wrap around the appendage and attached with an adhesive material. Due to perspiration and other factors such as fluid seepage from a wound, current methods for protection and healing of the area fall off. Further, long distance runners or individuals that excessively walk many miles or days are prone to callus and blisters. Current cushioning dressings for prevention of blisters and callus wrap around the toes connecting with an adhesive. Due to perspiration and other factors, current methods for protection and healing of the area fall off. Many patients due to disease such as diabetes and or trauma such as a burn have several toes in need of dressing separately. Current methods for dressing the toes individually involve wrapping each toe with gauze. This method is difficult and the gauze often adheres to the wound. It is a major goal of the present dressing and method to provide cushioning, support, as well as protection and healing for fingers and toes individually.

There are myriad laparoscopic surgical procedures that require maintained visibility of areas of a patient. This has proved particularly challenging. Laparoscopic cameras used in surgical procedures become covered in blood and fat. In addition, the laparoscopic camera lens becomes fogged. The surgeon is required to retract the camera out of the patient during the surgical procedure to clean the lens with cloth or gauze. Another method for cleaning the camera involves the surgeon manipulating the lens against a body part of the patient.

While these methods may be acceptable in some circumstances they cause prolonged surgical procedure time and increase the chance of complication. It is a major goal of the present invention to provide a dressing and method for cleaning a camera lens during a laparoscopic and or robotic surgical procedure.

Accordingly, there has been need for an improved method for cleaning the laparoscopic camera lens to maintain visibility of areas of a patient during a laparoscopic and or robotic surgical procedure. Further, there has been a need for an improved dressing and method to protect and clean a stoma, appendage, as well as the insertion sties of a catheters and or tubes in patients.

Examples of existing dressings and methods for cleaning laparoscopic instruments as well as for the protection and cleaning of stomas and insertion sites of catheters and tubes are seen in U.S. Pat. No. 2,327,514, U.S. Pat. No. 3,556,096, U.S. Pat. No. 3,683,991, U.S. Pat. No. 3,713,445 U.S. Pat. No. 3,811,436, U.S. Pat. No. 3,977,407, U.S. Pat. No. 4,122,857, U.S. Pat. No. 4,463,757, U.S. Pat. No. 4,865,594, U.S. Pat. No. 4,960,594, U.S. Pat. No. 4,969,880, U.S. Pat. No. 5,135,519, U.S. Pat. No. 5,221,265, U.S. Pat. No. 5,338,315, U.S. Pat. No. 5,616,116, U.S. Pat. No. 5,973,221, U.S. Pat. No. 6,019,996, U.S. Pat. No. 6,384,293, U.S. Pat. No. 6,779,525, U.S. Pat. No. 7,775,998, U.S. 2007/0135783, U.S. 2007/0161936, U.S. 2007/0027434, U.S. Patent 2009/0216168, U.S. Pat. No. 5,549,543, U.S. Pub No. 2007/0282253, U.S. Pat. No. 6,354,992, U.S. Pat. No. 5,464,008 and U.S. Pat. No. 5,313,934. U.S. 2010/012046.

### SUMMARY AND OBJECTIVES OF THE INVENTION

It is an object of the current dressing and method to overcome the disadvantages of the prior art by providing an improved dressing and method for cleaning laparoscopic equipment during laparoscopic and or robotic surgery as well as and improved dressing and method for protecting and cleaning stoma sites, appendages of the body as well as insertion sites of catheters and tubes.

In a more specific embodiment an exemplary dressing and method for protecting and cleaning a stoma, appendage, and the insertion area of tubes and catheters includes placing an annular shaped medical grade polyurethane hydrophilic foam or sponge like ring, made of an absorbent macro-bacterial flexible material designed to absorb fluid and or waste, circular in shape varying in outer and inner circumference, made with or without cross cuts in the center, or cuts along the side, varying in length, color, width and thickness, made or used with or without anti-bacterial agents and or fragrance, made or used with or without a sealant around the outer parameter of the dressing, around the stoma or an appendage of a patient, or around the catheter and or tube in a patient at the insertion site, as a way to cushion the area and provide protection from external trauma, provide a protective seal with the memory of the foam ring, and protection of the skin at the stoma and or insertion site preventing bacteria from growing, as well as breakdown of a patients skin around the stoma and insertion site of a catheter and tube.

Further, in a more specific embodiment an exemplary dressing and method for cleaning a laparoscopic camera lens intra-abdominally during a laparoscopic and or robotic surgery includes placing a sterilized annular shaped foam ring, containing an x-ray detectable element, made of an absorbent medical grade polyurethane hydrophilic or similar foam material, circular in shape, varying in length, color, width and thickness, around a surgical instrument that is then introduced into the abdominal cavity through prior art, positioning it adjacent to the laparoscopic camera, manipulating the foam ring against the laparoscopic camera lens cleaning and defogging the camera lens, or by grasping the present invention with prior art, introducing it into the body, then releasing it in the body, to be grabbed at its center using prior art, then manipulated against the laparoscopic camera lens against cleaning and defogging the camera lens, to be removed out of the body or left in the body for further cleaning during the procedure.

It is a major goal of the invention when used with prior art to add to the useful life of the ostomy appliances for colostomy, ilieostomy, urostomy, and tracheostomy patients, by placing the dressing around the aforementioned devices and or stomas. Prior art has a common disadvantage lacking a cushioning for comfort, which causes irritation. Prior art lacks a protective seal to protect the stoma site and surrounding area from infection, which the subject dressing would provide. Further, the subject dressing and method would provide for absorption of human waste and or fluids around the stoma, as well as prevent fluids from entering the stoma site preventing skin and tissue breakdown.

It is a major goal of the invention when used by placing around an abdominal stoma to provide cushioning and protection from environmental stressors such as pressure from a car safety belt that goes over an abdominal stoma. Prior art is bulky and cumbersome to wear and there is no comfort provided or protection from environmental stressors.

Further, it is also major goal of this invention to improve the method of cleaning a stoma area by placing the subject dressing around the stoma, then by manipulating the dressing against the skin with whatever needed degree of pressure needed, in a circular direction and or back and forth if needed, as to allow the dressing to be introduced into the crevice between skin and stoma, then removing the dressing slowing upward and away from the skin.

It is a goal of this invention to provide a patient with additional protection for their skin at the insertion sites of tubes and catheters by placing the dressing around the tube or catheter at the insertion site where the tube and or catheter enters the body providing a protective seal given that fluids excreting from or can seep into the insertion site. Further, by placing the subject invention modified with a sealant around the outer parameter, it prevents fluids from being absorbed into it and then into the insertion site.

In particular, by placing the subject dressing around a tube up to the insertion site of a nasal gastric tube, the secretions expelled from the nasal passage are absorbed into the dressing allowing the tube to stay in place as prior art does not provide.

It is also the goal of this invention to provide a patient cushioning, and comfort at the insertion site of catheters and tubes by placing the dressing around the tube or catheter up to the insertion site given its absorbing of environmental stresses. In particular, when used around a Foley catheter, which is introduced into the bladder, it provides the support to prevent pulling and tugging of the tube at the insertion site, which is a disadvantage of prior art. Prior art attaches the catheter to the leg. When the leg is moved or the patient walks the catheter is pulled causing pressure in the bladder, for the male his prostate, and irritation around the insertion site. Further, in the male patient, prior art is at a disadvantage as it causes a greater amount of skin touching such as penis and leg, which causes skin breakdown. A dome shaped dressing would cover the penis protecting the skin around the insertion site of the Foley catheter and the leg.

It is also major goal of this invention to improve the method of cleaning the skin at the insertion site of catheters and tubes by placing the dressing around the tube or catheter up to the insertion site, then by manipulating it against the skin with whatever needed degree of pressure, in a circular direction and or back and forth if needed, to be introduced into the crevice between skin and tube or catheter, then removing it away from the skin.

It is also a goal of this invention to improve the method of protecting and healing appendages such as toes and fingers by placing the subject dressing around a toe or finger. A disadvantage of prior art is that adhesive agents connecting two ends of a rectangular shape dressing fall off when moist or used with creams or lotions.

It is also a goal of this invention to overcome the disadvantages of the prior art by providing an improved method and dressing for cleaning a laparoscopic camera lens during laparoscopic and or robotic general surgery. A common disadvantage of prior art is that they are bulky, complicated and not cost effective.

### BRIEF DESCRIPTION OF THE DRAWINGS

The object of the invention will be better understood from the detailed description of its preferred embodiments which follows below, when taken in conjunction with the accompanying drawings, in which like numerals refer to like features throughout. The following is a brief identification of the drawing Figures used in the accompanying detailed description.

FIG. 1 shows in frontal view an ostomy patient 1, using prior art.

FIG. 1A shows in frontal view an ostomy patient 1, using prior art.

FIG. 2 shows a side view of a patient 1 using the current invention 9 with prior art.

FIG. 3 shows a side view of a patient 1 using the current dressing and method 9.

FIG. 4 shows in frontal view a tracheotomy patient 11 using prior art with the current invention 9.

FIG. 4A shows in a frontal view of a tracheotomy patient 11 with prior art using the subject dressing and method for cleaning 9.

FIG. 5 shows a side view of a patient with a nasogastric tube 17 using the invention 9.

FIG. 6 shows in perspective view a patient with a Foley catheter 20 using the invention 9.

FIG. 6A shows in perspective view a patient with a Foley catheter 20 using the subject dressing and method for cleaning 9.

FIG. 7 shows a frontal view of a patient with a gastric feeding tube 22 using the subject invention 9.

FIG. 7A shows a frontal view a patient with a gastric feeding tube 22 using the subject dressing and method for cleaning 9.

FIG. 8 shown in a perspective view a patient using the invention 9 around a rectal tube 23.

FIG. 9 shows a frontal view of the invention 9 around an appendage 6b.

FIG. 10 shows modifications of the invention 9.

FIG. 11 shows cross-sectioned view of a modification of the invention 9 incorporating a dome.

FIG. 12 illustrates a view of a modification of the invention 9 incorporating a cut along one side.

FIG. 13 illustrates a view of a modification of the invention in incorporating cross-cuts in the center.

FIG. 14 shows in frontal view the invention 9 used with surgical prior art.

FIG. 15 illustrates a prospective view of the invention 9 used with surgical prior art.

FIG. 16 shows a modification of the invention 9 with a sealant.

### DETAILED DESCRIPTION OF THE PREFERRED EMBODIMENTS

Certain exemplary embodiments will now be described to provide an overall understanding of the principals of structure, function, manufacture, and use of the dressing and method disclosed herein. One or more examples of these embodiments are illustrated in the accompanying drawings. Those skilled in the art will understand that the dressing and method specifically described herein and illustrated in the accompanying drawings are non-limiting exemplarily embodiments and that the scope of the present invention is defined solely by the claims.

The present invention generally provides for a dressing and method for cleaning a laparoscopic camera lens during laparoscopic and or robotic surgery intra-abdominally, as well as cushioning, protection and a method for cleaning a stoma, appendage, and the insertion site of catheters and tubes in patients.

The use of invention around a stoma, appendage, or at the insertion site of a patient, is of critical concern with respect to the comfort of a patient, protection of his or her skin and tissue from breakdown and infection, as well as a method of cleaning a stoma and or insertion site of a catheter and or tube to prevent infection.

The use of invention used with laparoscopic equipment intra-abdominally is of critical concern providing such benefits as improved visibility for a surgeon during laparoscopic and or robotic surgery shorting procedure time, which reduces chances for complications.

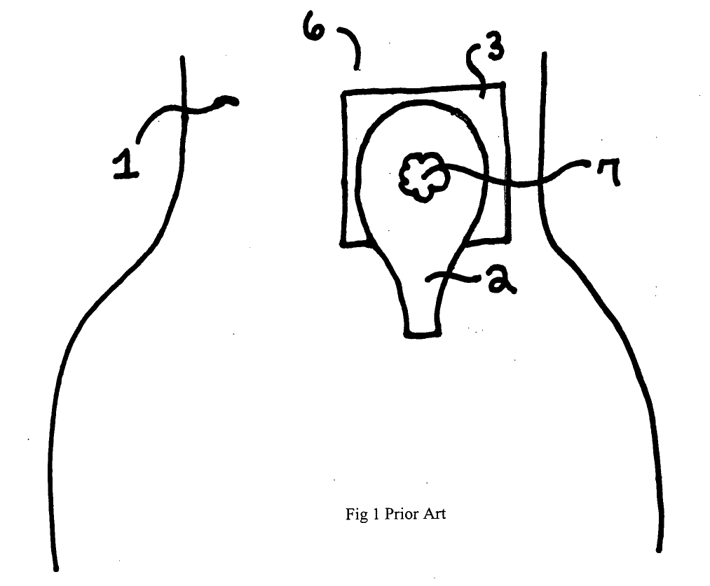

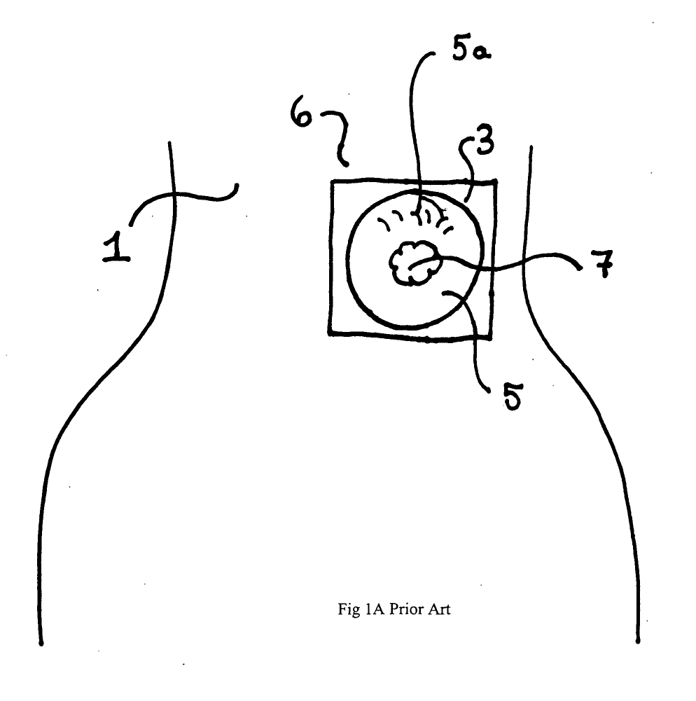

FIG. 1 shows a frontal view of an ostomy patient 1 using prior art such as ostomy bag 2 that would be attached to an ostomy appliance 3, which is attached to the skin of the patient 6. Prior art is at a disadvantage for the seal around the bag 2 and attachment site of the appliance 3 seeps human waste from a stoma 7 inside the bag.

FIG. 1A shows a frontal view of an ostomy patient 1 using prior art 5 that would be attached to an ostomy appliance 3, which is attached to the skin of the patient 6. Prior art is at a disadvantage for the vents 5a of the prior art 5 and seal where the prior art 5 is attached to the appliance 3 seeps human waste and gas from the stoma 7 beneath the prior art 5.

According to the device and method described herein, the absorbency of the material of the subject dressing and it's design would provide cushioning and support, absorb acid leakage from the stoma site 7, underneath the ostomy bag 3, as in FIG. 1 and prior art 5 in FIG. 1A away from the patient's skin 6. Further, the absorbency of the material of the subject dressing and its design would absorb gas and odor expelled from the ostomy site 7 in FIGS. 1 and 1A. The overall comfort of the patient is thereby increased. The subject dressing can be discarded when changing the ostomy appliance or when cleaning the stoma.

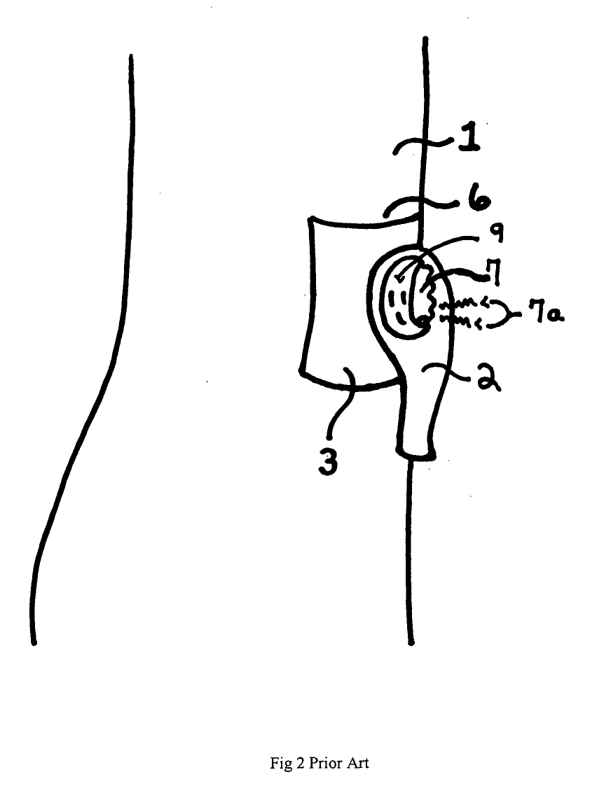

FIG. 2 is a side view of an ostomy patient 1 using the dressing 9 around the stoma 7 to collect gas and human waste 7a inside the colostomgy bag 2 protecting the stoma 7 from environmental stress, further providing cushioning and support, preventing the skin 6 of the patient 1 to breakdown thus increasing the life of the appliance 3. If the subject dressing 9 is used with a fragrance, the odor of the human waste 7a will be masked and provide the patient 1 with less discomfort and embarrassment.

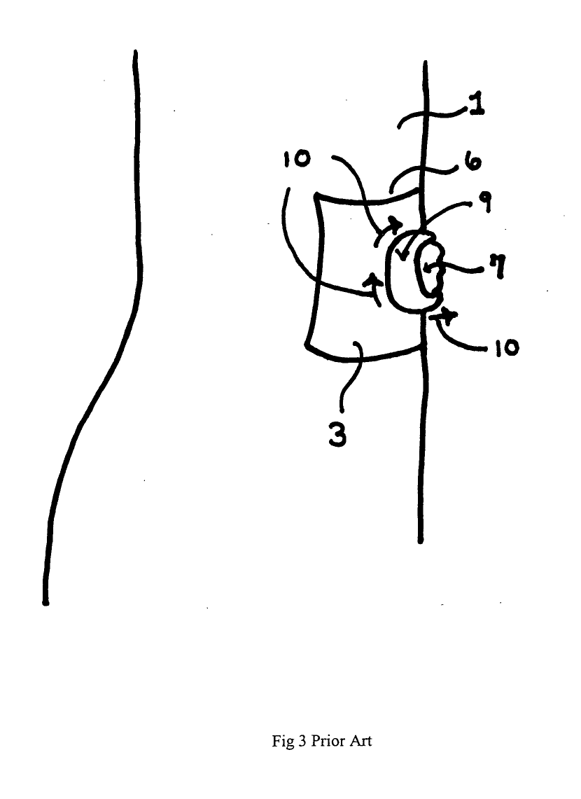

FIG. 3 illustrates an example of a method for cleaning the stoma site of a patient using the present invention. Manipulating the invention 9 against the skin 6 around the stoma 7 with whatever needed degree of pressure, in a circular direction, 10 back and forth 10, if needed, as to allow it be introduced into the crevice between skin 6 and stoma 7.

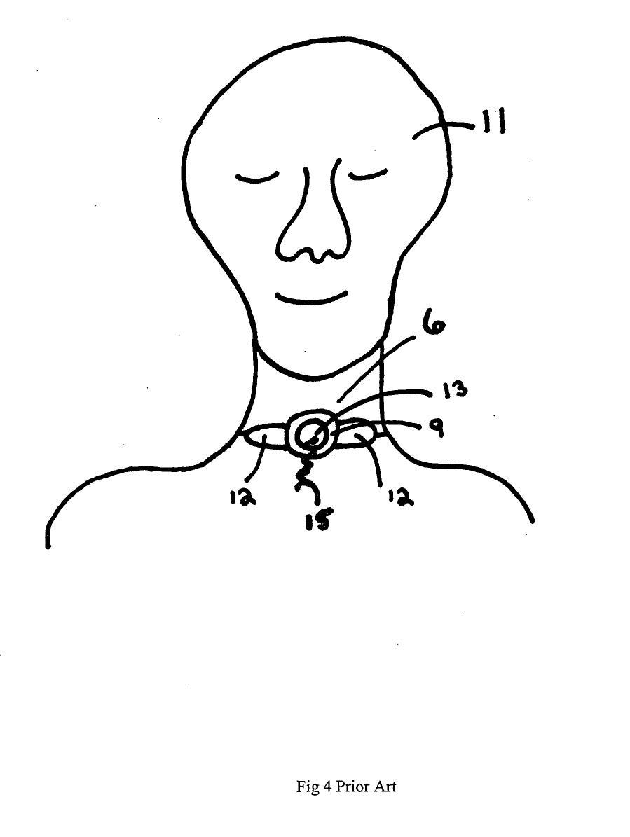

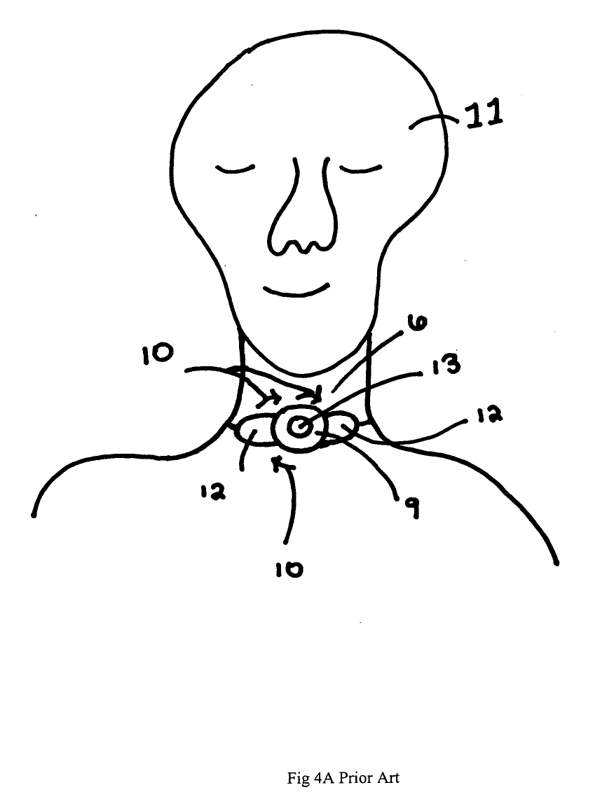

FIG. 4 illustrates an example of the device for use with a tracheotomy patient 11. The invention 9 is placed around prior art 12 that is inserted into and or around the stoma 13, pulling fluid 15 away from the skin 6 of a patient 11. The invention 9 provides additional cushioning and skin protection when used with prior art 12.

FIG. 4A demonstrates a method of use of the invention by manipulating the device 9 around the stoma 13 and prior art 12 in a tracheotomy patient 11 against the skin 6 with whatever needed degree of pressure, in a circular direction 10 back and forth 10 if needed, as to allow it to be introduced into the crevice between skin 6 and stoma 13.

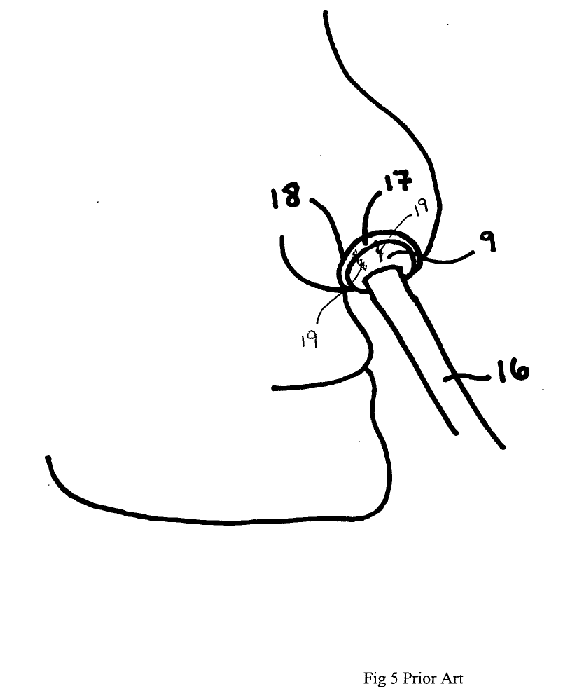

FIG. 5 illustrates an example of the invention being used with a patient that has had a nasogastric tube 16 inserted into the nasal cavity 17. By placing the invention 9 at the insertion site 18 the invention 9 absorbs the nasal secretions 19, further providing cushioning and protection from external trauma, keeping the nasal gastric tube 16 in the nasal cavity 17.

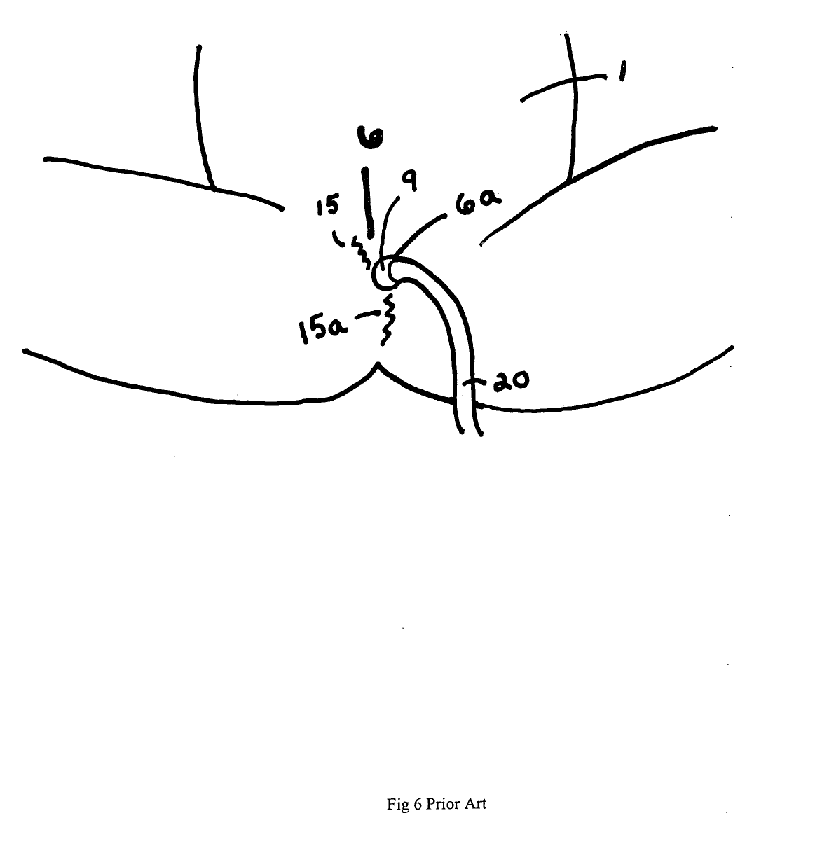

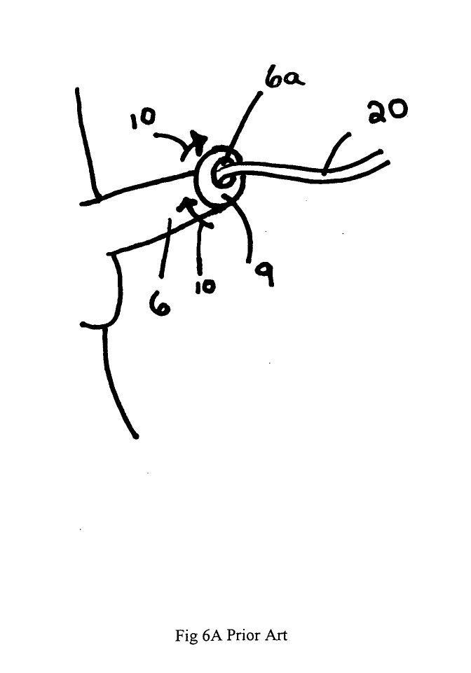

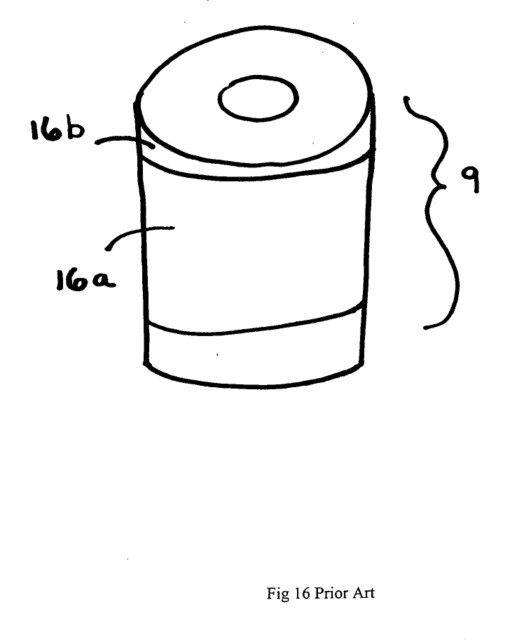

FIG. 6 shows a patient using the invention 9 around a Foley catheter 20 at the insertion site 6a, and skin 6 providing cushioning and protection at the insertion site 6a of the patient's skin 6. In addition to the invention providing the patient cushioning at the insertion site 6a of the catheter 20 the invention 9 has absorbent quality that pulls fluid 15 away from the skin 6 reducing the chance of infection. If desired, the foam of the invention 9 can be modified as in FIG. 16 with a sealant 16a as to prevent any fluid 15a as in FIG. 6 from entering the insertion site 6a.

FIG. 6A demonstrates a the subject method of the invention 9 by manipulating the subject dressing 9 around the catheter 20 at the insertion site 6a of a patient 1 against the skin 6 with whatever needed degree of pressure, in a circular direction 10, back and forth 10, if needed, as to allow it to be introduced into the crevice between skin 6 at the insertion site 6a and catheter 20.

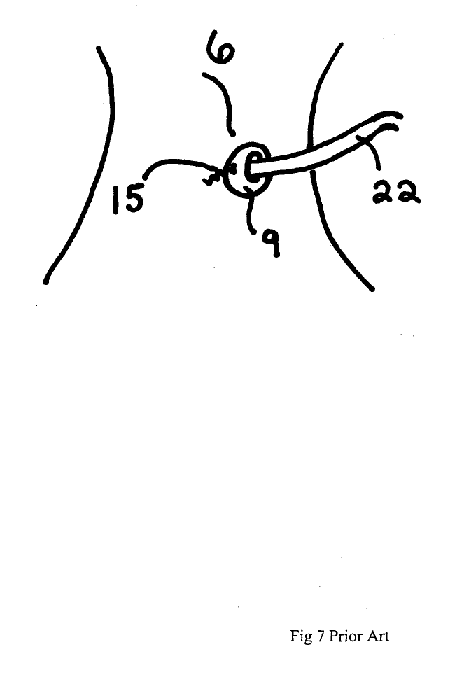

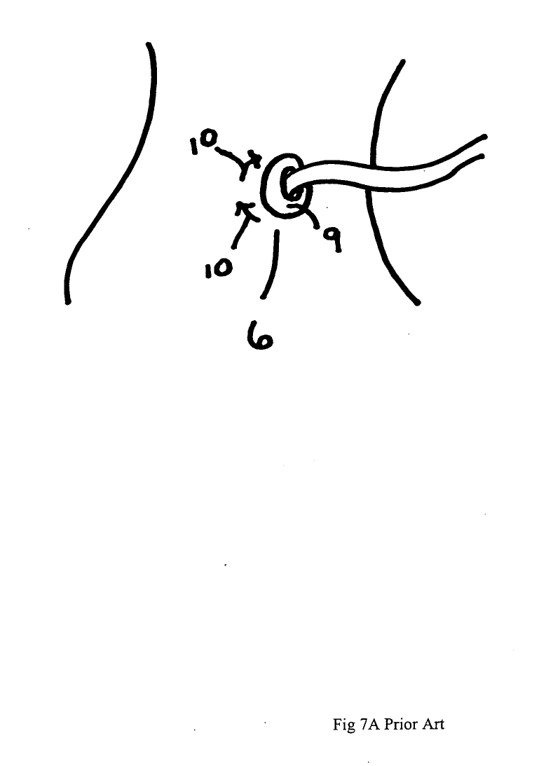

FIG. 7 shows a patient 1 using the invention 9 around a gastric feeding tube 22 at the insertion site 6a, and skin 6 providing a method of cushioning and protection at the insertion site 6a of the patient's skin 6. In addition to the invention providing the patient cushioning at the insertion site 6a of the tube 22, the invention 9, has an absorbent quality that pulls fluid 15 away from the skin 6 of the patient 1 reducing the chance of infection.

FIG. 7A demonstrates the subject method of use of the subject invention by manipulating the subject dressing 9 around the tube 22 at the insertion site 6a of a patient 1 with whatever needed degree of pressure, in a circular direction, 10 back and forth 10 if needed, as to allow it to be introduced into the crevice between skin 6 at the insertion site 6a and tube 22.

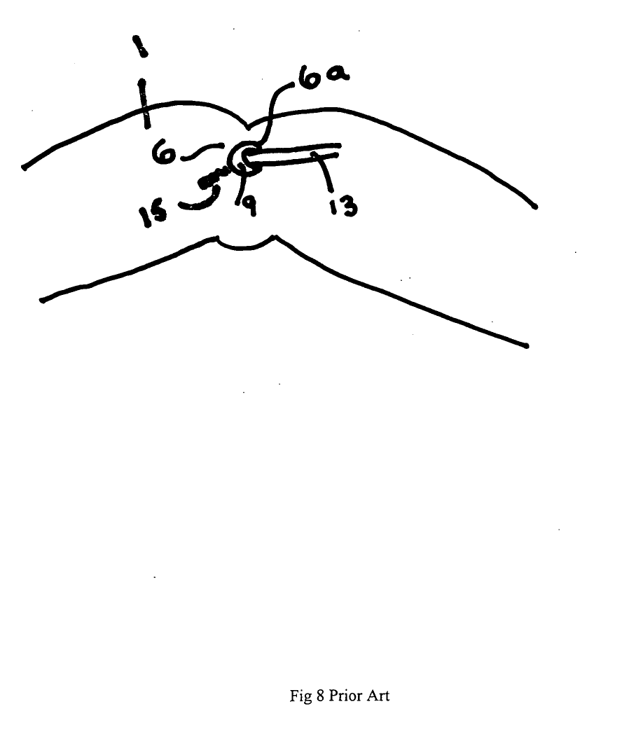

FIG. 8 shows a patient 1 using the subject invention 9 around a rectal tube 23 at the insertion site 6a, and skin 6 providing cushioning and protection at the insertion site 6a of the patient's 1 skin 6. In addition to the invention 9 providing the patient cushioning at the insertion site 6a of the tube 23, the invention 9, has absorbent quality that pulls fluid 15 away from the skin 6 of the patient 1 reducing the chance of infection and skin and tissue breakdown.

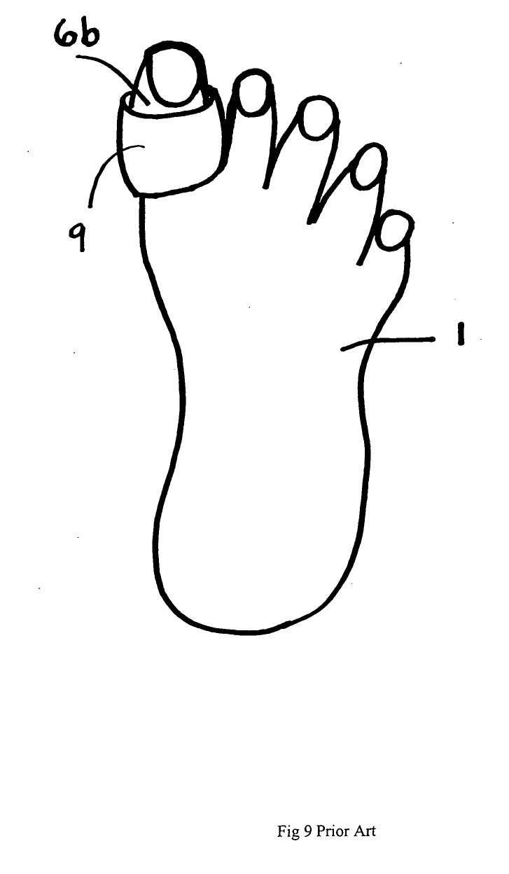

FIG. 9 shows the invention 9 used around the appendage 6b of a patient 1. The dressing can be used on appendages such as a toe or finger to promote wound healing from burns, blisters, tissue breakdown and cuts providing cushioning and absorbency preventing callus formation.

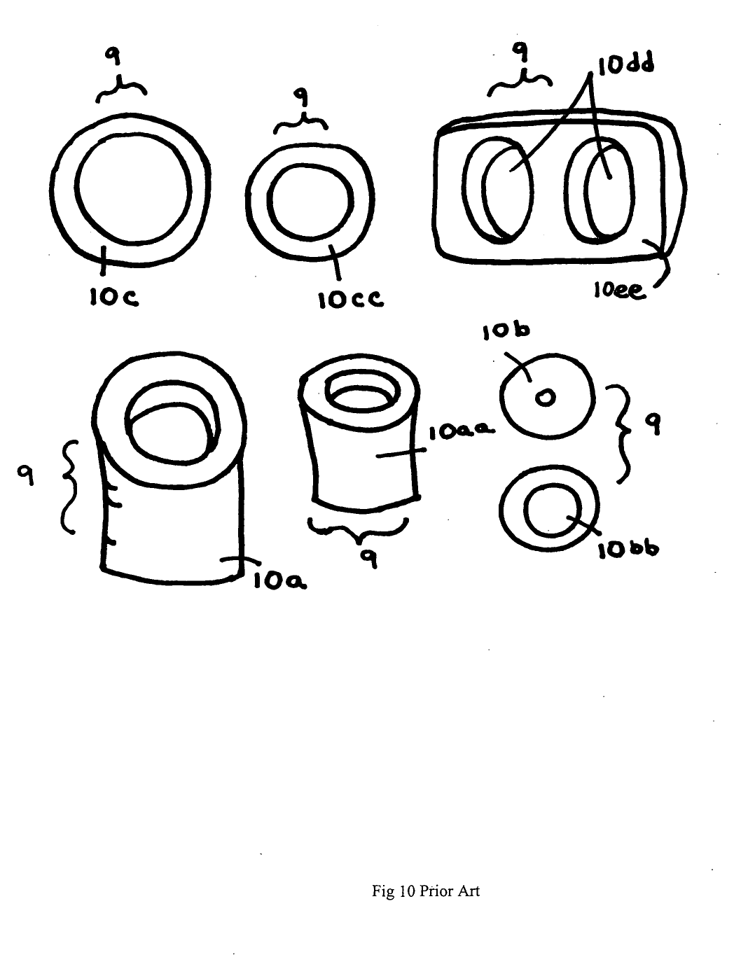

FIG. 10 illustrates a modification of the invention 9 that show a variation in length 10a 10aa, inner circumference opening 10b 10bb, outer circumference 10c 10cc and number of rings placed 10dd in a rectangular shape 10ee of the invention 9 for placing two or more appendages 6b as seen in FIG. 9. The modifications of the invention 9 can be custom tailored to the individual patients, stoma, toe or finger size.

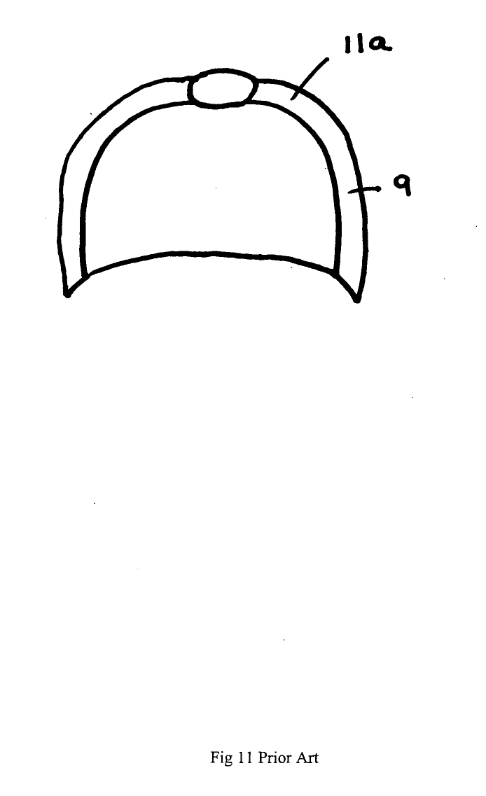

FIG. 11 illustrates a cross sectioned view of a modification of the invention 9 that is designed with a dome shape 11a at one end.

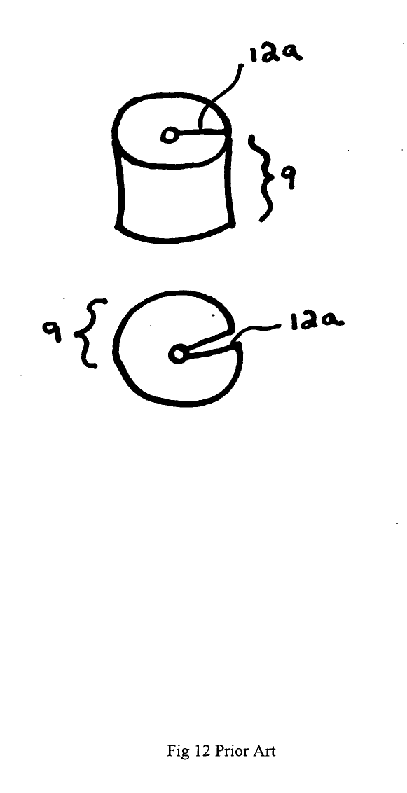

FIG. 12 and illustrates a view of a modification of the invention 9 incorporating a cut down one side 12a.

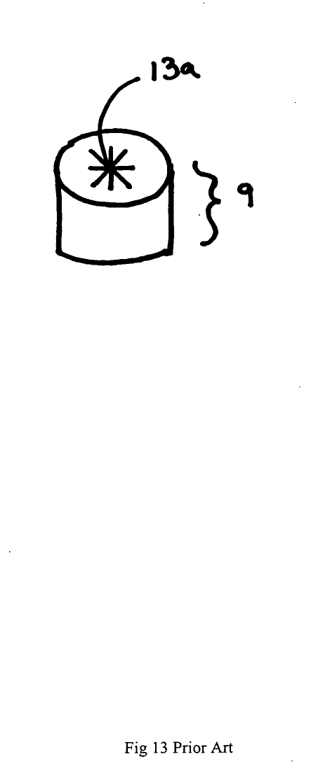

FIG. 13 illustrates a view of a modification of the invention 9 incorporating cross-cuts 13a in the center for use with smaller tubes as seen in FIGS. 6 and 6A.

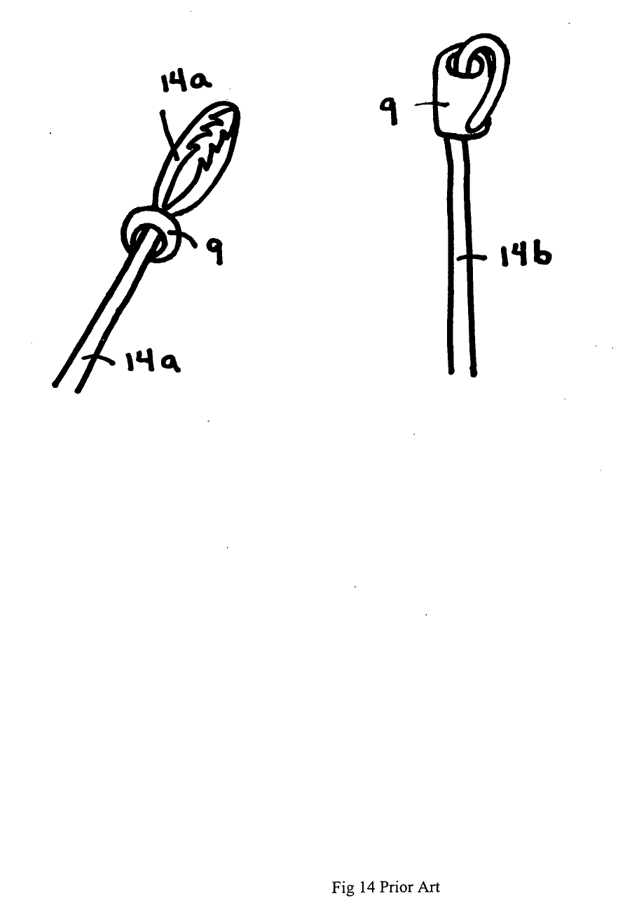

FIG. 14 shows in frontal view the invention 9 placed or glued around surgical prior art 14a and a frontal view of the invention 9 placed and or glued around surgical prior art 14b.

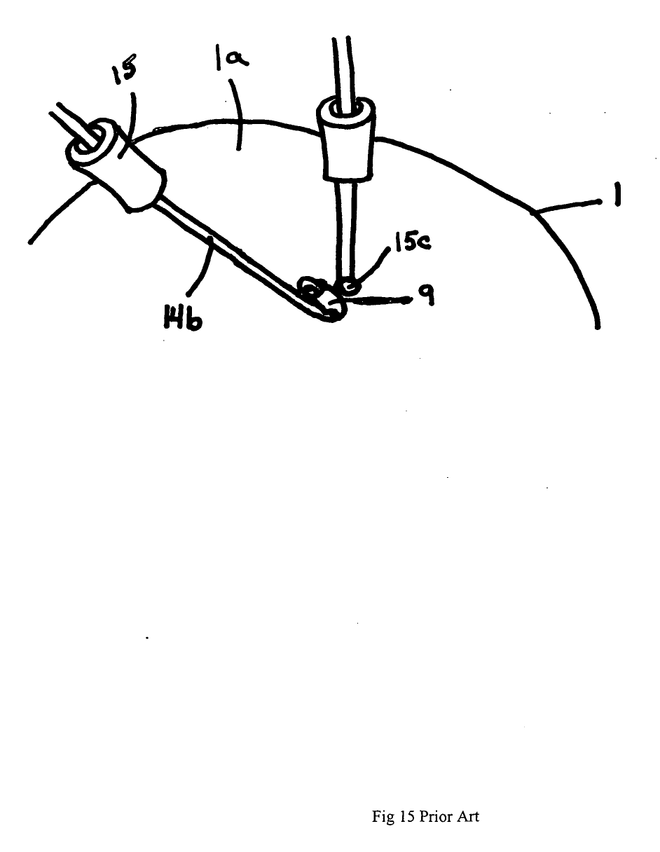

FIG. 15 illustrates a prospective view of the invention 9 used with prior art 14b as prior art 14b is inserted in the abdominal cavity 1a of a patient 1 during laparoscopic and or robotic surgery through prior art 15 manipulating the invention 9 against a laparoscopic camera lens prior art 15c, cleaning and defogging the lens, to be removed in the same manner as inserted.

FIG. 16 shows a modification of the invention 9 incorporating a sealant 16a around the outer parameter 16b of the invention 9.

The present invention as seen in FIGS. 3-16, consists in a typical embodiment of an annular shaped medical grade polyurethane hydrophilic or similar quality foam or sponge like ring, which is absorbent and flexible as to absorb fluid or waste material, circular in shape varying in outer and inner circumference, made with or without cross-cuts in the center, or cuts along the side, varying in length, color, width and thickness, made or used with or without anti-bacterial agents, x-ray detectable elements, fragrance, as well as a sealant around the outer parameter of the dressing.

One skilled in the art will appreciate that the subject dressing and method will create improvement to the cleaning of a laparoscopic camera lens during a laparoscopic and or robotic surgical procedure, as well as the comfort, healing, and prevention of infection for patients with an ostomy or those that have catheters and tubes inserted into their bodies, increasing the life of ostomy appliances, catheters and tubes, saving the patient and or healthcare provider money.

Further, one skilled in the art will appreciate the further features and advantages of the present invention based on the above-described embodiments providing comfort and cushioning while promoting healing of wounds, cuts, burns, and blisters around appendages such as toes and fingers.

What is claimed is:

1. A dressing including:
An annular tubular shape made of a moisture-absorbent flexible medical grade polyurethane hydrophilic or similar foam or sponge like material, and
circular in shape varying in outer and inner circumference size, and
made with or without cross-cuts in the center or cuts along the side, and
made with or without x-ray detectable elements, and
varying in length size and or color, and
made with or without a sealant on the outer rim, and
made with or without fragrance, and
made with or anti-bacterial agents.

2. A method for cleaning a stoma or the insertion site of a tube or catheter in a patient effectively with little irritation to the patient preventing blunting and further tissue damage to the skin.

3. A method as in claim 2 for providing a protective seal around the stoma and at the insertion site of tubes and catheters in patients from environmental trauma.

4. A method as in claim 2 for providing absorption of human waste and fluids around and in a stoma and at the insertion site of tubes and catheters in patients.

5. A method as in claim 2 for preventing human fluid, bacteria and or infection from seeping in a stoma and or at the insertion site of tubes and catheters in patients.

6. A method as in claim 2 for prevention of skin and tissue breakdown around the skin of a stoma in a patient by placing the invention as in claim 1 around the stoma.

7. A method as in claim 2 for prevention of skin and tissue breakdown around the insertion site of tubes in a patient by placing the invention as in claim 1 around the tube.

8. A method as in claim 2 for prevention of skin and tissue breakdown around the insertion site of catheters in a patient by placing the invention as in claim 1 around the catheter.

9. A method as in claim 2 for providing cleaning of human waste and fluids around and a stoma of a patient by placing the invention as in claim 1 around the stoma, manipulating the dressing as in claim 1 against the skin of a patient in a circular motion away from the skin of a patient.

10. A method as in claim 2 for providing cleaning of human waste and fluids around, in and at the insertion site of tubes in patients by placing the invention as in claim 1 around the tube, manipulating the foam ring against the skin of a patient at the insertion site of a tube in a circular motion away from the skin of a patient.

11. A method as in claim 2 for providing cleaning of human waste and fluids around, in and at the insertion site of catheters in patients by placing the invention as in claim 1 around the catheter, manipulating the foam ring against the skin of a patient at the insertion site of a catheter in a circular motion away from the skin of a patient.

12. A method as in claim 2 for providing wound care around an appendage such as a toe or finger by placing the invention as in claim 1 around the appendage.

13. A method as in claim 2 for providing cushioning and support around an appendage such as a toe or finger by placing the invention as in claim 1 around an appendage.

14. A method for cleaning a laparoscopic camera lens intra-abdominally during laparoscopic and or robotic surgery by placing or gluing the dressing as in claim 1 around prior art and then into the abdominal cavity of a patient manipulating the dressing in claim 1 against a laparoscopic camera lens cleaning and defogging the laparoscopic camera lens.

15. A method as in claim 14 for cleaning a laparoscopic camera lens intra-abdominally during laparoscopic and or robotic surgery by using prior art to grasp the invention at its center placing the invention into the abdominal cavity of a patient then manipulating the invention against a laparoscopic camera lens cleaning and defogging the laparoscopic camera lens removing the invention or leaving in the abdominal cavity against an organ of the body for further use.
